(19) United States
(12) Patent Application Publication
(10) Pub. No.: US 2007/0088257 A1
(43) Pub. Date: Apr. 19, 2007

(54) Rapid exchange catheter with hypotube and short exchange length

(75) Inventors: Beau M. Fisher, Danville, CA (US); Peter S. Brown, Palo Alto, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/251,236

(22) Filed: Oct. 13, 2005

(51) Int. Cl. A61M 29/00; A61M 31/00; A61M 37/00

(52) U.S. Cl. 604/103.04

(57) ABSTRACT

A rapid exchange balloon catheter includes a short rapid exchange portion which reduces friction between the guidewire and the catheter. The catheter shaft includes one or more sections of hypotubing and overjacketing of diminishing distal flexibility by, e.g., forming cuts in the hypotubing and/or varying the jacketing material and/or thickness. A polymer jacket overlies the hypotubing, and an inner sleeve is provided adjacent to the proximal guidewire port to further strengthen the catheter shaft.

### BACKGROUND OF THE INVENTION

1. Field of the Invention

The invention relates to interventional catheters, and more particularly to rapid exchange balloon catheters.

2. Brief Description of the Related Art

Rapid exchange balloon catheters are described in U.S. Pat. Nos. 4,762,129 and 5,040,548, the entireties of which are incorporated herein by reference. These rapid exchange catheters include a distal guidewire lumen which extends through the balloon from a distal end of the balloon to a guidewire exit port that is proximal of the balloon, but which is distal of the proximal end of the catheter. In these and other known rapid exchange balloon catheter systems the catheter shafts include a proximal stiff catheter section extending along about 75% of the catheter length and a distal more flexible portion of the catheter between the stiff section and the balloon. For catheters which are designed for angioplasty or for stent delivery, the portion of the catheter proximal of the balloon and distal to the stiffer proximal catheter section should be simultaneously very flexible to navigate the coronary arteries, have good column strength to provide pushability, and have good kink resistance. The proximal catheter section generally requires good column strength and less flexibility.

Solid wires or hypotubes have been used for the proximal sections of rapid exchange catheters due to their excellent pushability and small diameter. However, the hypotubes and wires which have been used do not have the flexibility required for the distal flexible portion of the catheter.

U.S. Pat. No. 4,762,129, granted to Bonzel, has a short guidewire lumen with the guidewire exiting the catheter shaft just proximal of the balloon. The short exchange length of the Bonzel catheter can provide improved ease and speed of use for the user. The Bonzel catheter has drawbacks, however, including the lack of guidewire support of the distal portion of the catheter shaft resulting in possibilities of kinking and lack of pushability.

Therefore, there is a need for an improved rapid exchange catheter with a short exchange length without sacrificing any of the performance features provided by a longer exchange length.

### SUMMARY OF THE INVENTION

According to a first aspect of the invention, a rapid exchange catheter is comprised of a proximal catheter shaft formed of a hypotube, a distal catheter shaft formed of a hypotube, an expandable balloon attached to the distal catheter shaft; and a guidewire lumen extending from a distal end of the balloon, through an interior of the balloon to a proximal guidewire inlet adjacent a proximal end of the balloon. The distal catheter hypotube extends to within 3 cm of the proximal guidewire inlet.

According to another aspect of the invention, a rapid exchange catheter is comprised of a catheter shaft formed of a hypotube of a nickel titanium alloy, an expandable balloon attached to a distal end of the catheter shaft and a guidewire lumen extending from a distal end of the balloon, through an interior of the balloon to a proximal guidewire inlet adjacent a proximal end of the balloon. The catheter hypotube extends to within 3 cm of the proximal guidewire inlet.

According to a further aspect of the invention, a catheter is comprised of a first hypotube of a first metal, wherein a distal portion of the first hypotube is cut to provide a change in flexibility from a less flexible proximal end to a more flexible distal end, a second hypotube of a second metal, the second hypotube being more flexible than the first hypotube, wherein a distal portion of the second hypotube is cut to provide a change in flexibility from a less flexible proximal end to a more flexible distal end, a connection between the distal end of the first hypotube and the proximal end of the second hypotube, and a polymer jacket extending over the cut portion of the first and second hypotubes.

According to another aspect of the invention, a balloon catheter is comprised of a stainless steel proximal hypotube, a nickel titanium distal hypotube connected to the proximal hypotube, a cut portion at a distal end of the nickel titanium distal hypotube which provides increased distal flexibility of the catheter and an expandable balloon connected to the distal end of the nickel titanium hypotube. A distance between the spiral cut portion and the balloon is about 40 mm or less.

According to a further aspect of the invention, a balloon catheter is comprised of a stainless steel proximal hypotube, a nickel titanium distal hypotube connected to the proximal hypotube, a cut portion at a distal end of the nickel titanium distal hypotube which provides increased distal flexibility of the catheter and an expandable balloon connected to the distal end of the nickel titanium hypotube. A distance between the spiral cut portion and the balloon is about 40 mm or less.

According to another aspect of the invention, a balloon catheter is comprised of a nickel titanium hypotube having a cut distal end for increased distal flexibility, a polymer jacket surrounding the cut distal end and an expandable balloon connected to the distal end of the nickel titanium hypotube. A distance between the laser cut distal end and the balloon is about 40 mm or less.

According to a further aspect of the invention, a rapid exchange catheter is comprised of a proximal catheter shaft formed of a hypotube having a first outer diameter, a distal catheter shaft formed of a hypotube having a second outer diameter, an expandable balloon attached to the distal catheter shaft, and a guidewire lumen extending from a distal end of the balloon, through an interior of the balloon to a proximal guidewire inlet adjacent a proximal end of the balloon. The second outer diameter is less than the first outer diameter.

According to another aspect of the Invention, a method of performing a vascular procedure in a mammalian patient comprises the steps of inserting a catheter into the vasculature of the patient and moving the catheter to a location in the vasculature of the patient. The catheter includes a proximal catheter shaft formed of a hypotube, a distal catheter shaft formed of a hypotube, an expandable balloon attached to the distal catheter shaft and a guidewire lumen extending from a distal end of the balloon, through an interior of the balloon to a proximal guidewire inlet adjacent a proximal end of the balloon. The distal catheter hypotube extends to within 3 cm of the proximal guidewire inlet and moving the catheter to a location in the vasculature of the patient.

Still other aspects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to embodiments of the apparatus and method exemplifying principles of the invention, given only by way of example, and with reference to the accompanying drawings, in which:

FIG. 1 illustrates a side perspective view of a catheter system;

FIG. 2 illustrates a side elevational, longitudinal cross-sectional view, with central portions broken away, of the catheter illustrated in FIG. 1.

FIG. 3 illustrates a side elevational, longitudinal cross-sectional, enlarged view of a portion of the catheter illustrated in FIG. 1, according to a first exemplary embodiment of the present invention;

FIG. 4 illustrates a side elevational, longitudinal cross-sectional, enlarged view of a portion of the catheter illustrated in FIG. 1, according to a second exemplary embodiment of the present invention;

FIG. 5 illustrates a side elevational, longitudinal cross-sectional, enlarged view of a portion of the catheter illustrated in FIG. 1, according to a third exemplary embodiment of the present invention;

FIG. 6 illustrates a side elevational, longitudinal cross-sectional, enlarged view of a portion of the catheter illustrated in FIG. 1, according to a fourth exemplary embodiment of the present invention; and

FIG. 7 illustrates a side elevational, longitudinal cross-sectional view, with central portions cut away, of catheter according to a fifth exemplary embodiment of the present invention.

### DETAILED DESCRIPTION OF EXEMPLARY EMBODIMENTS

Referring to the drawing figures, like reference numerals designate identical or corresponding elements throughout the several figures.

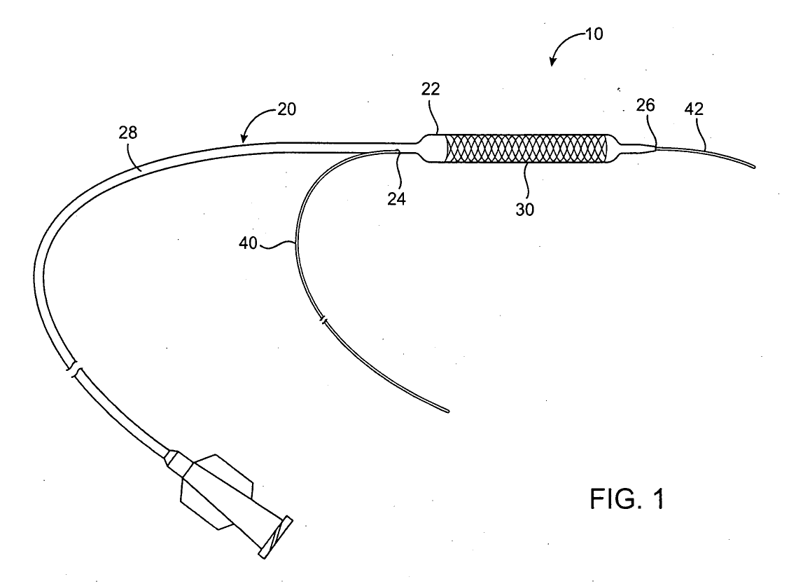

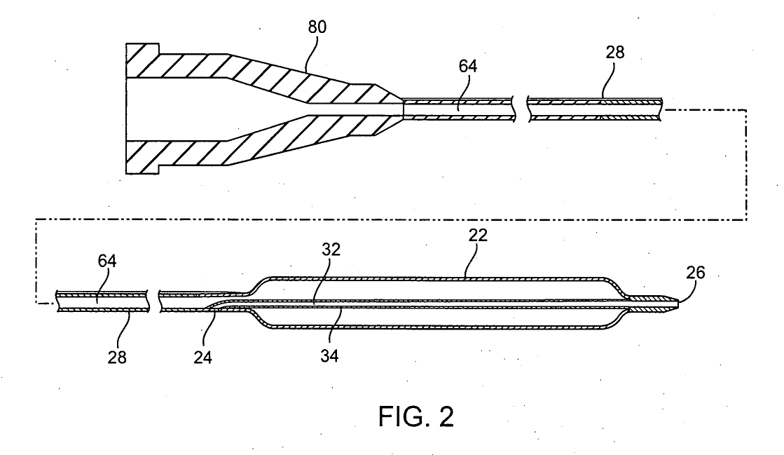

A catheter system 10 illustrated in FIG. 1 embodies principles of the present invention, and includes a catheter 20 (shown schematically), which is optionally a rapid exchange angioplasty balloon catheter, a stent 30, and a guidewire 40. FIG. 1 illustrates the catheter 20 including an expandable balloon 22, with the balloon in an expanded or inflated configuration. In an unexpanded or delivery configuration, the balloon 22 and stent 30 will have an outer diameter close to the outer diameter of the shaft 28 of the catheter 20. As illustrated in FIG. 1, the catheter 20 includes a distal guidewire port 26 at a distal end of the catheter and a proximal guidewire port 24 proximal of balloon 22. According to an exemplary use, the catheter 20 is inserted into a patient, e.g., into the vasculature of a patient, over the guidewire 40 by passing the guidewire into the distal port 26 of the catheter, through the lumen 32 of a guidewire exchange tube 34 (see FIGS. 2-5) which passes through the balloon 22, and out the side opening of the proximal guidewire port 24 of the catheter, optionally leaving distal portions 42 of the guidewire extending distally of the catheter.

Because the details of stents and guidewires are well known to those skilled in the art, and because the present invention is not limited by such details, further description of the stent 30 and the guidewire 40 will not be given so as to not obscure the principles of the present invention.

FIG. 2 illustrates a side elevation, longitudinal cross-sectional view, with central portions broken away, of the catheter 20 illustrated in FIG. 1. The catheter shaft 28 extends between a proximal hub structure 80 distally to the balloon 22, and defines an inflation lumen 64 therein. A guidewire guide tube 34 extends from the proximal port 24, located proximal of the balloon 22, distally through the interior of the balloon, and to a location distal of the balloon to the distal port 26. The guide tube 34 defines an inner lumen 32 sized and shaped to permit the guidewire 40 to slide therethrough. Thus, the catheter 20 includes a relatively short rapid exchange portion, i.e., that portion of the catheter between ports 24 and 26, which can have numerous advantages as detailed elsewhere herein.

The shaft 28 advantageously includes a structure which changes the flexibility of the shaft. According to an advantageous embodiment of the present invention, a portion of the shaft includes one or more lengths of hypotubing, one or more overlayers or exterior jackets, or both. While several exemplary embodiments of a catheter 20 including hypotubing will now be described, the present invention is not limited to the details of these embodiments.

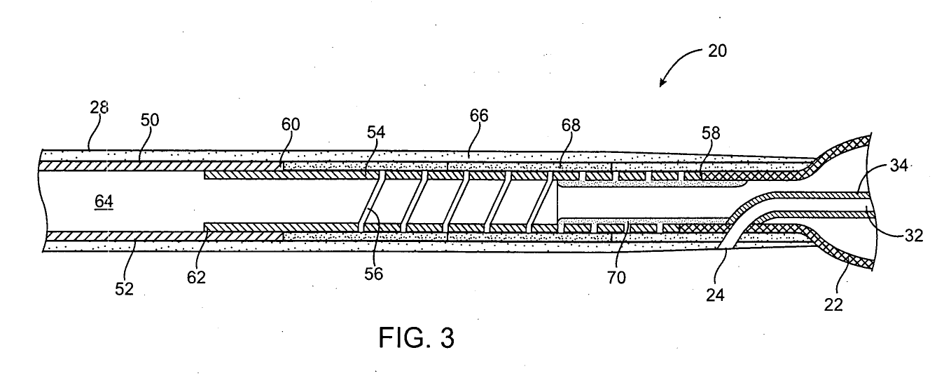

FIG. 3 illustrates a side elevational, longitudinal cross-sectional, enlarged view of a portion of the catheter 20 illustrated in FIG. 1, according to a first exemplary embodiment of the present invention. The catheter shaft 28 includes a hypotube shaft 50 including a proximal portion 52 and a distal portion 54, with the proximal 52 and distal 54 portions being joined together near the distal end 60 of the proximal portion and the proximal end 62 of the distal portion. The inflation lumen 64 extends through the hypotube shaft 50 into the interior of the balloon 22. In the exemplary embodiment of FIG. 3, the two portions of the hypotube 50 are joined at a telescoping connection, that is, the distal end 60 of the proximal portion 52 is positioned distally of the proximal end 62 of the distal portion 54, and one of the two portions has an outer dimension, e.g., diameter for a circular cross-sectional shape, that is less than the inner dimension, e.g., diameter, of the other portion. In the embodiment illustrates in FIG. 3, the distal portion 54 is positioned inside the proximal portion 52; the invention also includes the reverse, although not illustrated, i.e., the proximal portion being positioned within the distal portion. According to yet another embodiment, the junction or transition between a less flexible proximal portion and a more flexibie distal portion can be formed in a single-piece, monolithic hypotube by reducing the outer diameter of the more distal portion, e.g., by necking down the distal portion, or by expanding the proximal portion, or both, of the single hypotube.

While the exemplary embodiments described herein refer to two hypotube portions, the present invention is not so limited, and extends to a monolithic hypotube having features described elsewhere herein to modify its flexibility, and extends to a hypotube including more than two portions.

Another advantageous, optional feature of the present invention includes that the hypotube 50 has one or more cuts 56 formed therein, which increases the flexibility of the hypotube in the area of the cut. By way of example and not of limitation, the shape of the cut can be helical, longitudinally slotted, laterally slotted, as well as other shapes and orientations of one or more slots, so that the flexibility of the hypotube increases distally. Additionally, the slot 56, or the density of slots, can be non-uniform, and advantageously can be formed so that the configuration of the slot contributes to the increasing flexibility of the shaft 28. By way of non-limiting example, a helical slot, such as that illustrated in FIG. 3, can be formed, e.g., by laser cutting, with an increasing pitch of the helix; thus, there is more of the cut 56 per unit length of the hypotube distally, and is thus more flexible distally. Similarly, longitudinal cuts (not illustrated) can have larger widths or simply more cuts distally, and lateral (for a circular cross-section, circumferential) cuts can be spaced more closely together distally.

The distal end of each portion of the hypotube 50 is designed, e.g., by selecting a cut configuration, outer diameter and wall thickness of the hypotube, and the like, so that the flexibilities of the distal and of a proximal portion is similar to that of the proximal end of a distal portion. That is, where two portions of the hypotube meet, there is not a significant increase or decrease in the flexibility of the hypotube 50.

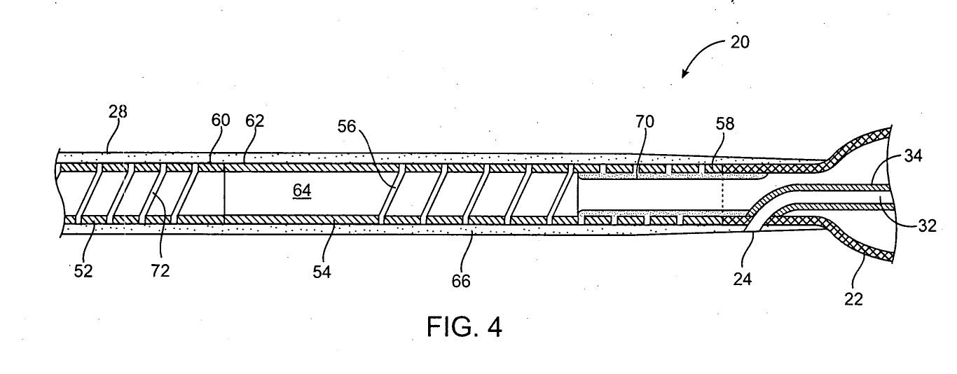

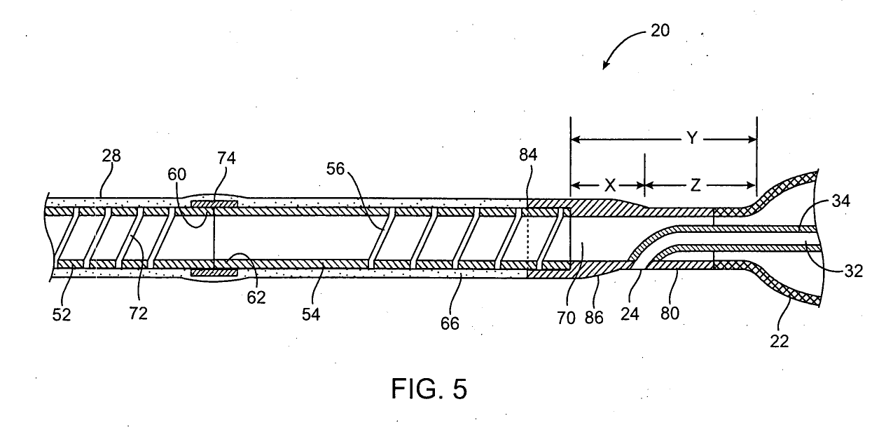

Yet another advantageous, optional feature of the present invention is the further inclusion of an outerjacket 66 over the hypotube 50. By forming the jacket 66 of a biocompatible material, e.g., a biocompatible polymer, the catheter shaft 28 can be made fluid tight to the inflation fluid passing through inflation lumen 64, relatively low friction to assist in passing the catheter 20 through the vasculature of a patient, and the flexibility of the catheter shaft 28 can further be modified. More specifically, the jacket 66 can be formed of a material, and having thicknesses, so that the flexibility of the shaft 28 increases distally. In the embodiment illustrated in FIG. 3, because the junction of the proximal portion 52 and the distal portion 54 of the hypotube 50 involves a change in the outer diameter of the hypotube, a second, inner jacket 68 is also provided to smooth out the transition between the two portions. As with jacket 66, jacket 68 can be designed to increase the flexibility of the shaft 28 distally, such as by providing the jacket 68 in several sections with different flexibilities, as suggested in FIG. 3. Alternatively, jacket 68 can be a part of and integral with jacket 66. Furthermore, in the alternative arrangement in which the distal portion 54 of the hypotube 50 has a larger OD than that of the proximal portion 52, jacket 68 can be positioned around a part or all of the proximal portion of the hypotube. Further optionally, the jacket 66 can be formed in segments. While the embodiments illustrated herein include a jacket 66 which extends over more than the portions of the hypotube 50 that include cuts 56, 72 (see, e.g., FIGS. 4, 5), the present invention is not so limited, and also includes providing the jacket over only the cut portions of the hypotube.

Another advantageous, optional feature of the present invention is that the cut 56 in the hypotube portion(s) terminates before reaching the distal end of that portion, leaving a ring of uncut material at the distalmost part of that hypotube portion. With reference to the exemplary embodiment illustrated in FIG. 4, which also illustrates an alternative, butt weld junction between the proximal and distal portions 52, 54, of the hypotube 50, the distal end 60 of the proximal portion 52, and the distal end 58 of the distal portion 54, do not include the cut 72, 56, respectively, therein. Providing a distal end part of each portion of the hypotube without the cut permits better bonding between the hypotube portion and other structures, as well as prevents a cut end of the hypotube from puncturing out of the catheter shaft 28.

Turning back to FIG. 3, the catheter shaft 28 further can include an inner polymer sleeve 70 on the inner surface of the hypotube 50 immediately adjacent to and proximal of the location of the proximal guidewire port 24. The further provision of the sleeve 70 provides yet another structure in the shaft 28 the composition and dimensions of which can be selected to modify the flexibility of the shaft distally. The sleeve 70 also strengthens the area of the catheter 20 to aid in bonding the guidewire tube 34, the balloon 22, and the hypotube 50, and to increase the pressure capabilities of the catheter in this region. The inner sleeve 70 is bonded in place, in an exemplary embodiment, by inserting a tapered mandrel prior to formation of the surrounding structures of the rapid exchange bond.

Turning now to FIG. 5, another exemplary embodiment of a catheter 20 is illustrated, in which the junction between the proximal portion 52 and the distal portion 54 of the hypotube 50 includes an outer sleeve 74. The sleeve 74 can be bonded, welded, or crimped over the hypotube portions to secure the portions together. Preferably, as illustrated in FIG. 5, the jacket 66 overlies the sleeve 74, providing a smooth exterior surface. Further optionally, or in addition, the sleeve 74 can be positioned on the inner surface of the hypotube 50. Each of the hypotube portion junctions of the present invention are configured so that, like the ends of the portions themselves, there is not a significant increase or decrease in the flexibility at the junction.

In the exemplary embodiment illustrated in FIG. 5, the distal hypotube portion 54 is joined with a bonding tube 80, which is in turn joined with the proximal portion of the balloon 22. The bonding tube 80 has a flexibility designed in accordance with the principles of the present invention, and is preferably at least as flexible as the distal portions of the distal hypotube portion 54 to which it is joined. The bonding tube can be formed of any one or more of numerous materials, although a biocompatible polymer material is preferred. As illustrated in FIG. 5, the guidewire guide tube 34 extends proximally into the bonding tube 80, in which the proximal port 24 is formed.

Somewhat different from other embodiments described herein, the jacket 66 terminates before the distal end of the distal hypotube portion 54 at a point 84, and the bonding tube 80 is bonded over the end of the distal portion 54 and bonded to the jacket 66. The bonding tube 80 is formed of a material that will, when melted, blend into and with the material of the jacket 66 and the balloon 22. Thus, while FIG. 5 illustrates a distal terminus 84 of the jacket 66, preferable embodiments include a blending of the jacket 66 and the tube 80 in this region.

Another advantageous, optional feature of the present invention includes that the flexibility of the shaft 28 is designed to increase from proximal portions of the shaft toward the distal end, so that the shaft is more flexible distally. One exemplary embodiment of the present invention includes a proximal portion 52 which is more rigid, less flexible, than a distal portion 54. This change in flexibility in the shaft 28 can be achieved according to the present invention through one or more of the following: using different materials for the different portions of the hypotube; changing the outer diameter (OD) of the shaft to be smaller distally; changing the composition of the polymer jacket material(s), thickness of the polymer jacket, and number of layers of polymer jackets; and the pattern(s) of the cuts in the hypotubing.

Yet another advantageous, optional feature of the present invention includes one or more coatings on the inner, outer, or both inner and outer, surfaces of the catheter shaft 28. The coatings on the catheter shaft are selected to provide lubricity, drug delivery, and/or other beneficial characteristics to the catheter 20, as well understood and appreciated by the skilled artisan.

According to a yet further advantageous embodiment, proximal portions of the hypotube 50, e.g., the proximal portion 52, are formed of a stainless steel, while distal portions of the hypotube, e.g., the distal portion 54, is formed of a superelastic material, e.g., a nickel titanium alloy, e.g., NiTi. The dimensions and exact alloy materials of the two portions are selected so that the flexibility of the hypotube 50, and the shaft 28, increases distally, as described elsewhere herein. Stainless steel hypotubes extending all the way to the balloon 22 may be less preferable, because they may experience permanent deformation or kinking. A NiTi distal hypotube portion can eliminate the permanent deformation problem, because of its known deformation behavior. While a NiTi shaft extending all the way from the proximal end to distal end can also be used in the present invention, it is also less preferably because of the increased cost of that solution. Alternatively, a NiTi portion can be replaced with a low residual stress stainless steel tubing, or another superelastic material, known well by those of skill in the art.

The cut, jacketed hypotubes translate forces well in compression, translate some forces in tension, and transmit torque very well. Torquability is important to allow the practitioner, e.g., physician, to navigate the catheter through tight lesions or tight turns in the patient's vasculature by rotating the catheter. Pushability, or the ability to transmit forces in compression, and is sometimes measured by comparing the amount of force the physician must apply at the proximal end to get a given force at the distal end. The short rapid exchange length of catheters in accordance with the present invention provides improved trackability because the short contact length between the guidewire lumen and the guidewire provides less friction than a longer guidewire lumen.

The proximal guidewire port 24 can further optionally include a longitudinal groove on the exterior of the shaft 28, such as that described in co-assigned provisional application No. 60/684,775, filed 26 May 2005, entitled “Rapid exchange balloon catheter with reinforced shaft”, by Beau M. Fisher et al., the entirety of which is incorporated by reference herein.

According to a particularly preferred embodiment, the distalmost end of the distal end of the hypotube 50 is a distance X from the proximal guidewire port 24, or is a distance Y from the proximal end of the balloon 22, or both; wherein X is between about 0.05 cm and about 5 cm, preferably less than 3 cm, more preferably less than 2 cm, most preferably between 0.1 cm and 0.3 cm; and wherein Y is between about 0.1 cm and about 10 cm, preferably between about 0.1 cm and about 4 cm, more preferably less than about 3 cm, most preferably between 0.6 cm and 1 cm. It is also useful for understanding principles of the present invention to define the distance Z=Y−X, the distance from the proximal end of the balloon 22 to the proximal guidewire port 24. Another aspect of the present invention is, therefore, dimensioning portions of the catheter 20 so that Z is quite short, that is, the rapid exchange tube 34 extends very little proximally of the proximal end of the balloon 22. For example, Z is preferably about 0.1 cm to about 5 cm, more preferably about 0.2 cm to about 1.5 cm.

In the foregoing description, the flexibility of the catheter shaft 28 has been described as increasing distally. According to a somewhat less preferred embodiment of the present invention, but still within the scope of the present invention, one or more sections of the catheter shaft 28, including but not limited to the one or more junctions between hypotubing portions, may have flexibilities that are less than portions that are proximal thereto. That is, the present invention extends to the optional inclusion in the catheter shaft 28 of some portions of the catheter shaft that are stiffer than in embodiments in which the flexibility of the catheter shaft does not decrease distally. In this context, the present invention also includes embodiments in which the flexibility of the catheter shaft does not change over the length of some portions, and also includes more flexible distal portions and, as described above, less flexible portions.

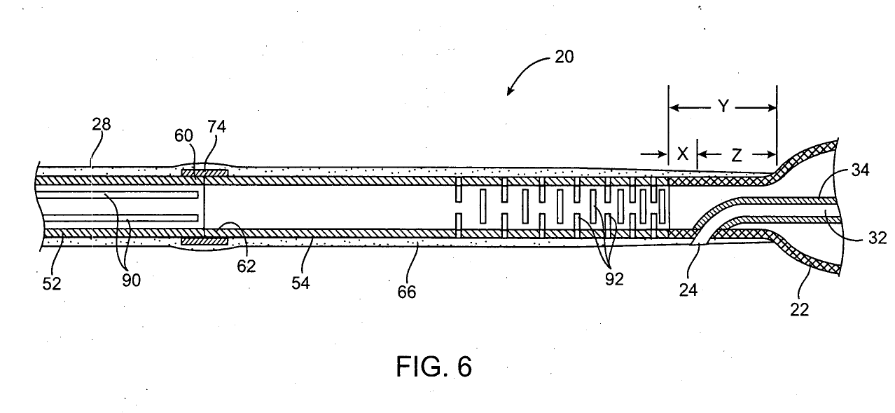

FIG. 6 illustrates yet another exemplary catheter 20 embodying principles of the present invention. In some respects, the embodiment illustrated in FIG. 6 is similar to other embodiments described herein, and therefore the structures and functions common thereto will not be repeated. In the embodiment illustrated in FIG. 6, longitudinal cuts 90 and lateral (or, for circular cross sectional elements, circumferential) cuts 92 are formed in the hypotube portions, to modify the flexibility of the hypotube in these portions. More specifically, either or both of the proximal hypotube portion 52 and the distal hypotube portion 54 can include longitudinal cuts 90 or lateral cuts 92, instead of the spiral cuts previously described herein. Further optionally, the size, number, and spacing of the cuts 90, 92 can be variable so that the flexibility of the hypotube portion in which the cut is formed can be selectively modified. By way of non-limiting example, the number of longitudinal cuts 90 can increase distally, the width of one or more of the longitudinal cuts can increase distally, or combinations thereof, to increase the flexibility of the hypotube portion in which the cuts are formed. By way of further non-limiting example, the number of lateral cuts 90 can increase distally, as suggested in FIG. 6 by the increased frequency of the cuts, distally, the width of one or more of the lateral cuts can increase distally, or combinations thereof, to increase the flexibility of the hypotube portion in which the cuts are formed. In this regard, it is preferable that lateral cuts are spaced so that the hypotube portion is not completely severed.

For both longitudinal 90 and lateral cuts 92, it is preferable that the cuts are formed so that the flexibility of the particular hypotube portion is substantially the same in every lateral direction, that is, the cuts are formed so that the particular hypotube portion has essentially the same flexibility in all radial directions taken from the centerline or longitudinal axis of that hypotube portion.

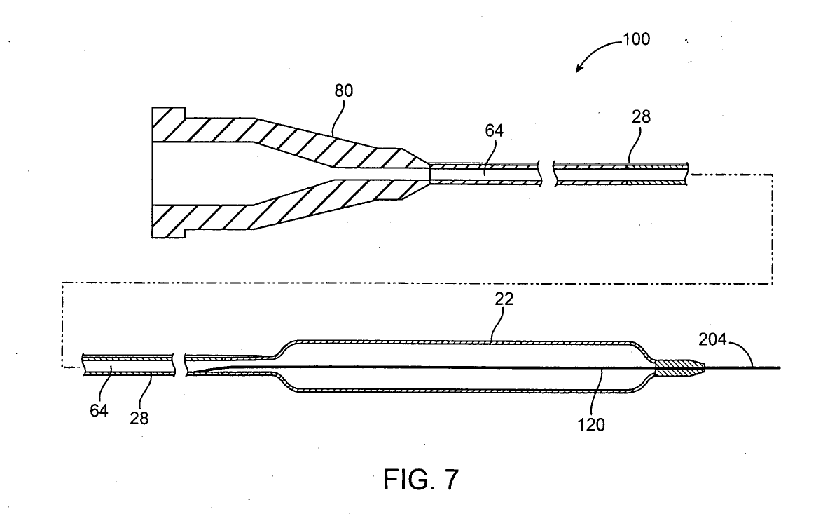

Although the foregoing describes aspects of the present invention in the context of a rapid exchange balloon angioplasty catheter, the present invention is not limited to such devices. Accordingly, additional embodiments exemplifying principles of the present invention include rapid exchange and non-rapid exchange catheters, balloon and non-balloon catheters including, but not limited to, infusion catheters, angiography catheters, thermal and/or RF and/or laser ablation catheters, and fixed-wire vascular catheters. With reference to FIG. 7, a fixed-wire catheter 100 is illustrated, in accordance with the present invention. The embodiment illustrated in FIG. 7 is, in some respects, similar to the exemplary embodiment illustrated in FIG. 2, and therefore similar structures and functions will not be further repeated. The catheter 100 includes a fixed guidewire 120 that extends from the hypotube 28 of the catheter 100, through the inflation lumen 64, through the balloon 22, and to a distal guidewire tip 204. As those of ordinary skill in the art are well acquainted with the construction of steerable, fixed wire catheters, further embellishment of catheter 100 will not be provided herein so as not to obscure the principles of the present invention. The catheter 100 includes one or more of the other structures described with reference to FIGS. 3-6 herein.

With reference to the drawing figures, an exemplary method embodying further principles of the present invention will now be described. A catheter in accordance with the present invention is inserted into the vasculature of a mammalian, preferably human, patient, optionally over a guidewire, and is advanced to a vascular location of interest. The balloon of the catheter may then be inflated or expanded in a manner well appreciated by the skill artisan, e.g., by increasing the pressure applied to an inflation fluid, and the balloon's diameter increases. When a stent is positioned on the exterior surface of the balloon, the stent is thus expanded, in a well know manner. Thus, the balloon and/or the stent can be expanded against the interior surface of the vascular vessel, which may include plaques, the vascular intima, and other structures.

While the invention has been described in detail with reference to exemplary embodiments thereof, it will be apparent to one skilled in the art that various changes can be made, and equivalents employed, without departing from the scope of the invention. Each of the aforementioned documents is incorporated by reference herein in its entirety.

What is claimed is:

1. A rapid exchange catheter comprising:
a proximal catheter shaft formed of a hypotube;
a distal catheter shaft formed of a hypotube;
an expandable balloon attached to the distal catheter shaft; and
a guidewire lumen extending from a distal end of the balloon, through an interior of the balloon to a proximal guidewire inlet adjacent a proximal end of the balloon, and wherein the distal catheter hypotube extends to within 3 cm of the proximal guidewire inlet.

2. The catheter of claim 1, wherein the proximal catheter hypotube and the distal catheter hypotube are formed from a single hypotube.

3. The catheter of claim 2, wherein the single hypotube is formed of a nickel titanium alloy.

4. The catheter of claim 1, wherein the distal catheter hypotube is formed of a nickel titanium alloy.

5. The catheter of claim 1, further comprising:
a polymer jacket covering at least a portion of the hypotube; or
wherein the distal catheter hypotube is cut to improve flexibility; or
both.

6. The catheter of claim 5, comprising a distal catheter hypotube cut in a spiral pattern.

7. The catheter of claim 6, wherein the spiral pattern changes in pitch to vary the flexibility of the distal catheter hypotube flexibility from less flexible at the proximal end of the distal catheter shaft to most flexible at the distal end of the distal catheter shaft.

8. The catheter of claim 6, wherein the spiral pattern cut in the distal catheter hypotube stops before the distal end of the distal catheter shaft providing a continuous ring of hypotube material at the distal end of the distal catheter shaft.

9. The catheter of claim 1, wherein the catheter is an angioplasty catheter.

10. The catheter of claim 1, further comprising:
a stent on exterior of the balloon.

11. The catheter of claim 1, comprising a polymer jacket having a flexibility that increases distally by a characteristic selected from the group consisting of:
a changing composition of the polymer jacket material;
a changing thickness of the polymer jacket;
a changing number of layers of polymer jacket; and
combinations thereof.

12. A rapid exchange catheter comprising:
a catheter shaft formed of a hypotube of a nickel titanium alloy;
an expandable balloon attached to a distal end of the catheter shaft; and
a guidewire lumen extending from a distal end of the balloon, through an interior of the balloon to a proximal guidewire inlet adjacent a proximal end of the balloon, and wherein the catheter hypotube extends to within 3 cm of the proximal guidewire inlet.

13. The catheter of claim 12, further comprising:
a polymer jacket covering at least a portion of the hypotube; or
wherein the distal catheter hypotube is cut to increase flexibility; or
both.

14. The catheter of claim 13, comprising a distal catheter hypotube cut in a spiral pattern.

15. The catheter of claim 14, wherein the spiral cut changes in pitch to vary the flexibility of the hypotube flexibility from less flexible at a proximal end of the spiral cut to most flexible at a distal end of the spiral cut.

16. The catheter of claim 14, wherein the spiral pattern cut in the hypotube stops before a distal end of the catheter shaft providing a continuous ring of hypotube material.

17. The catheter of claim 13, comprising a polymer jacket having a flexibility that increases distally by a characteristic selected from the group consisting of:
a changing composition of the polymer jacket material;
a changing thickness of the polymer jacket;
a changing number of layers of polymer jacket; and
combinations thereof.

18. A catheter comprising:
a first hypotube of a first metal, wherein a distal portion of the first hypotube is cut to provide a change in flexibility from a less flexible proximal end to a more flexible distal end;
a second hypotube of a second metal, the second hypotube being more flexible than the first hypotube, wherein a distal portion of the second hypotube is cut to provide a change in flexibility from a less flexible proximal end to a more flexible distal end;
a connection between the distal end of the first hypotube and the proximal end of the second hypotube; and
a polymer jacket extending over the cut portion of the first and second hypotubes.

19. The catheter of claim 18, further comprising:
an expandable balloon connected to the distal end of the second hypotube.

20. The catheter of claim 18, wherein the catheter is a rapid exchange catheter having an inflation lumen extending from the proximal end of the first hypotube to the distal end of the second hypotube, and a guidewire lumen exiting distal of the proximal end of the distal hypotube.

21. The catheter of claim 18, wherein the catheter is an angioplasty catheter.

22. The catheter of claim 18, wherein the polymer jacket has a distal end and a proximal end, a flexibility of the catheter between the distal end and proximal end of the polymer jacket varying by changing the polymer material or thickness along the length of the polymer jacket.

23. The catheter of claim 18, wherein the connection is formed by one of a butt weld, a lap joint, and a sleeve.

24. The catheter of claim 18, wherein the first hypotube includes a spiral cut.

25. The catheter of claim 24, wherein the spiral cut has a varying pitch to provide a change in flexibility.

26. The catheter of claim 24, wherein the spiral cut does not extend entirely to the distal end of the first hypotube, leaving a continuous ring of metal at the distal end of the first hypotube.

27. The catheter of claim 18, wherein the second hypotube includes a spiral cut.

28. The catheter of claim 27, wherein the spiral cut has a varying pitch to provide a change in flexibility.

29. The catheter of claim 27, wherein the spiral cut does not extend entirely to the distal end of the second hypotube, leaving a continuous ring of metal at the distal end of the second hypotube.

30. The catheter of claim 18, wherein the catheter is a rapid exchange balloon angioplasty catheter, and further comprising:
an expandable balloon; and
a rapid exchange guidewire port proximal of the balloon and distal of the second hypotube.

31. The catheter of claim 30, wherein a spacing between the distal end of the second hypotube and the proximal end of the balloon is about 4 cm or less.

32. The catheter of claim 18, wherein the first hypotube is formed of a stainless steel alloy and the second hypotube is formed of a nickel titanium alloy.

33. The catheter of claim 18, wherein the second metal is more flexible than the first metal.

34. The catheter of claim 18, wherein the outer diameter of the first hypotube is greater than the outer diameter of the second hypotube.

35. A balloon catheter comprising:
a stainless steel proximal hypotube;
a nickel titanium distal hypotube connected to the proximal hypotube;
a cut portion at a distal end of the nickel titanium distal hypotube which provides increased distal flexibility of the catheter; and
an expandable balloon connected to the distal end of the nickel titanium hypotube, wherein a distance between the spiral cut portion and the balloon is about 40 mm or less.

36. The catheter of claim 35, wherein the catheter is a rapid exchange catheter having an inflation lumen extending from the proximal end of the proximal hypotube to the distal end of the distal hypotube, and a guidwire lumen exiting distal of the proximal end of the distal hypotube.

37. The catheter of claim 35, wherein the catheter is an angioplasty catheter.

38. The catheter of claim 35, further comprising:
a polymer jacket extending over the cut portion of the distal hypotube,

39. The catheter of claim 38, wherein the polymer jacket has a distal end and a proximal end, a flexibility of the catheter between the distal end and proximal end of the polymer jacket varying by changing the polymer material or thickness along the length of the polymer jacket.

40. The catheter of claim 35, further comprising:
a connection between the proximal hypotube and the distal hypotube formed by one of a butt weld, a lap joint, and a sleeve.

41. The catheter of claim 35, wherein the cut is spiral shaped.

42. The catheter of claim 41, wherein the spiral cut has a varying pitch to provide a change in flexibility.

43. The catheter of claim 41, wherein the spiral cut does not extend entirely to the distal end of the distal hypotube, leaving a continuous ring of metal at the distal end of the distal hypotube.

44. The catheter of claim 35, wherein the proximal hypotube includes a spiral cut.

45. The catheter of claim 44, wherein the proximal spiral cut has a varying pitch to provide a change in flexibility.

46. The catheter of claim 44, wherein the proximal spiral cut does not extend entirely to the distal end of the proximal hypotube, leaving a continuous ring of metal at the distal end of the proximal hypotube.

47. The catheter of claim 35, wherein a spacing between the distal end of the distal hypotube and the proximal end of the balloon is about 4 cm or less.

48. A balloon catheter comprising:
a nickel titanium hypotube having a cut distal end for increased distal flexibility;
a polymer jacket surrounding the cut distal end; and
an expandable balloon connected to the distal end of the nickel titanium hypotube, wherein a distance between the laser cut distal end and the balloon is about 40 mm or less.

49. The catheter of claim 48, wherein the cut is a laser cut.

50. A rapid exchange catheter comprising:
a proximal catheter shaft formed of a hypotube having a first outer diameter;
a distal catheter shaft formed of a hypotube having a second outer diameter;
an expandable balloon attached to the distal catheter shaft;
a guidewire lumen extending from a distal end of the balloon, through an interior of the balloon to a proximal guidewire inlet adjacent a proximal end of the balloon; and
wherein the second outer diameter is less than the first outer diameter.

51. The catheter of claim 50, wherein at least one of the proximal catheter hypotube and the distal catheter hypotube is formed of a nickel titanium alloy.

52. The catheter of claim 50, wherein the distal catheter hypotube is formed of a nickel titanium alloy.

53. The catheter of claim 50, further comprising:
a polymer jacket covering at least a portion of the hypotube; or
wherein the distal catheter hypotube is cut to improve flexibility; or both.

54. The catheter of claim 53, comprising a distal catheter hypotube cut in a spiral pattern.

55. The catheter of claim 54, wherein the spiral cut changes in pitch to vary the flexibility of the distal catheter hypotube flexibility from less flexible at the proximal end of the distal catheter shaft to most flexible at the distal end of the distal catheter shaft.

56. The catheter of claim 54, wherein the spiral cut in the distal catheter hypotube stops before the distal end of the distal catheter shaft providing a continuous ring of hypotube material at the distal end of the distal catheter shaft.

57. The catheter of claim 50, wherein the catheter is an angioplasty catheter.

58. The catheter of claim 50, further comprising:
a stent on exterior of the balloon.

59. A method of performing a vascular procedure in a mammalian patient, comprising:
inserting a catheter into the vasculature of the patient, the catheter including
a proximal catheter shaft formed of a hypotube;
a distal catheter shaft formed of a hypotube;
an expandable balloon attached to the distal catheter shaft; and
a guidewire lumen extending from a distal end of the balloon, through an interior of the balloon to a proximal guidewire inlet adjacent a proximal end of the balloon, and wherein the distal catheter hypotube extends to within 3 cm of the proximal guidewire inlet;
moving the catheter to a location in the vasculature of the patient.

60. The method of claim 59, wherein inserting comprising inserting the catheter over a guidewire, the guidewire extending through the guidewire lumen.
